(19) United States
(12) Patent Application Publication
(10) Pub. No.: US 2014/0004504 A1
(43) Pub. Date: Jan. 2, 2014

(54) COMPOSITIONS AND METHODS FOR THE DETECTION AND ANALYSIS OF AFRICAN SWINE FEVER VIRUS

(71) Applicants: Lawrence J. Wangh, Auburndale, MA (US); Bonnie Ronish, St. Louis, MO (US)

(72) Inventors: Lawrence J. Wangh, Auburndale, MA (US); Bonnie Ronish, St. Louis, MO (US)

(73) Assignee: BRANDEIS UNIVERSITY, Waltham, MA (US)

(21) Appl. No.: 13/993,028

(22) PCT Filed: Dec. 9, 2011
(86) PCT No.: PCT/US11/64222

§ 371 Date: Sep. 19, 2013

Related U.S. Application Data
(60) Provisional application No. 61/421,772, filed on Dec. 10, 2010.

(51) Int. Cl. C12Q 1/70

(52) U.S. Cl. CPC C12Q 1/701
USPC 435/5; 536/24.33

(57) ABSTRACT

Provided herein are compositions and methods for the detection and analysis of African swine fever virus (ASFV). In particular, kits, compositions, and methods employing LATE-PCR reagents and processes for the detection and analysis of ASFV are provided.

### CROSS-REFERENCE TO RELATED APPLICATION

The present invention claims priority to U.S. Provisional Patent Application Ser. No. 61/421,772 filed Dec. 10, 2010, which is hereby incorporated by reference in its entirety.

### FIELD

Provided herein are compositions and methods for the detection and analysis of African swine fever virus (ASFV). In particular, kits, compositions, and methods employing LATE-PCR reagents and processes for the detection and analysis of ASFV are provided.

### BACKGROUND

African swine fever (ASF) is an economically important, highly lethal disease of domestic pigs that is listed as notifiable to the OIE (World Organization for Animal Health). It is caused by African swine fever virus (ASFV); previously classified as an iridovirus based on its morphology, it is now classified as the sole member within the family Asfarviridae (genus Asfivirus). ASFV is a cytoplasmic, double-stranded DNA virus with a linear, non-segmented genome 170 kb to 190 kb in length (Blasco et al., 1989b). It contains 151 to 165 open reading frames, depending on the strain (Blasco et al., 1989a; Kleiboeker et al., 2001).

African swine fever virus (ASFV) is a highly pathogenic hemorrhagic DNA virus that infects domestic pigs. The mortality rate from virulent hemorrhagic strains of ASFV often approaches 100% in domestic pigs, whereas infection with less virulent strains may result in sub-acute or chronic infections with lower mortality (Boinas et al. 2004; Dixon et al., 2004; ICTVdB, 2006). Currently there is no vaccine, treatment or cure for ASF, and infected animals, as well as those suspected of infection, are slaughtered.

The first documented outbreak of ASF occurred in Kenya in 1921 (Montgomery et al., 1921) and since then ASF has been reported in most countries of Sub-Saharan Africa, where the virus is maintained either through a sylvatic cycle involving warthogs and/or bush pigs, and soft ticks in the genus Ornithodoros, or in a domestic cycle that involves pigs of local breeds, with or without tick involvement (Anderson et al., 1998; Oura et al., 1998a; Kleiboeker, et al., 2001; Boinas et al., 2004). Since there is no currently available control measure other than diagnosis and slaughter, the disease poses a serious constraint to the development of both smallholder and industrial pig industries in Africa. Moreover, the disease poses a continuous threat to countries outside the African continent, as shown by major outbreaks in Portugal and Spain in the past, and the ongoing devastating epizootic in the Caucasus region which started in Georgia in 2007 (Rowlands et al., 2007). In the fall of 2009 the deadly disease of ASF jumped 2,000 kilometers from the Caucasus region to St Petersburg in north-western Russia (FAO report, http://www.fao.org/news/story/en/item/36622/icode/)

The clinical picture of ASF is virtually indistinguishable from that of classical swine fever (CSF) another OIE notifiable disease of swine. Diagnosis of both ASF and CSF therefore relies heavily on sophisticated testing (Agüero et al., 2004; Rodriguez-Sanchez et al, 2008). The ASFV genome is relatively conserved from strain to strain, with three main variable regions: a central region (CVR) of relatively high variability (Nix et al., 2006) and two terminal variable regions (Blasco et al., 1989a,b; Sumption et al., 1990). One gene in particular, the B646L gene that encodes the major capsid protein p72 (aka VP72) is very highly conserved across all strains (Bastos et al., 2003; Bastos et al., 2004).

Several methods have been previously described for the detection of ASFV (Malmquist et al., 1960; Alcaraz et al., 1990; Steiger et al., 1992; King et al., 2003; Agüero et al., 2004; Zsak et al., 2005; Hutchings et al., 2006; McKillen et al., 2007; Giammarioli et al., 2008). Most of these assays produce accurate results within twenty-four hours, including sample preparation and virus detection. Current pan-detection assays for ASFV target the VP72 gene based on its high level of conservation. The assay currently recommended by EU and OIE reference laboratories is a closed-tube, TaqMan® PCR assay developed by King et al. (2003) to detect a portion of the VP72 gene. This assay provides detection of ASFV DNA within 24 hours of sample receipt with an analytical sensitivity between 100 and 10 copies (King et al., 2003).

### SUMMARY

Provided herein are compositions and methods for the detection and analysis of African swine fever virus (ASFV). In particular, kits, compositions, and methods employing LATE-PCR reagents and processes for the detection and analysis of ASFV are provided.

In some embodiments, provided herein are methods for detecting or analyzing African swine fever virus (ASFV) in a sample, comprising: contacting a sample with reagents for performing amplification (e.g., LATE-PCR); amplifying ASFV nucleic acid from the sample to generate amplified ASFV nucleic acid; and detecting the amplified ASFV nucleic acid. In some embodiments, provided herein are kits for detecting or analyzing African swine fever virus (ASFV) in a sample, comprising: reagents for performing amplification (e.g., LATE-PCR) on ASVF nucleic acid. In some embodiments, the sample comprises an environmental sample. In some embodiments, the environmental sample is a water or soil sample. In some embodiments, the sample is biological sample. In some embodiments, the biological sample is taken from a pig (e.g., family Suidae, e.g., Sus domestica). In some embodiments, the biological sample is a tissue sample. In some embodiments, the biological sample is a fluid sample. In some embodiments, the sample comprises a mixture of biological samples from multiple organisms. In some embodiments, the ASFV nucleic acid is purified from the sample prior to amplification. In some embodiments, the ASFV nucleic acid is a strain of ASFV selected from the group consisting of Moz64, Ang72, MwLil 20/1, CV97, Ug03H, Ken06.B1, Ken07.Eld1, BF07, E70, Ba71V, E75, L60, Ss88, and Haiti. In some embodiments, the sample contains less than 10 copies of ASFV genome. In some embodiments, the reagents comprise amplification primers. In some embodiments, the amplification primers hybridize to ASFV VP72 gene. In some embodiments, the amplification primers comprise a limiting primer and an excess primer, wherein the limiting primer at its initial concentratoin has a melting temperature relative to a target sequence that is higher than or equal to the excess primer melting temperature relative to a the target sequence at its initial concentration, in accord with the teaching and theory of LATE-PCR (See, e.g., U.S. Pat. No. 7,198,897; herein incorporated by reference in its entirety). In some embodiments, the limiting primer comprises CTGATACGTGTCCATAAAACGCAGGTGAC (SEQ ID NO.:1), or a sequence having at least 70% identity therewith (e.g., greater than 80%, 90%, 95%). In some embodiments, the excess primer comprises CTGGAAGAGCTGTATCTCTATCCTG (SEQ ID NO.:2), or a sequence having at least 70% identity therewith (e.g., greater than 80%, 90%, 95%). In some embodiments, the reagents comprise a probe. In some embodiments, the probe is a molecular beacon. In some embodiments, the probe comprises a fluorescent label. In some embodiments, the probe has a melting temperature relative to a target nucleic acid that is lower than the melting temperature of an annealing step in an amplification reaction used in the amplifying. In some embodiments, the probe melting temperature is approximately 55° C. or lower. In some embodiments, the probe comprises AACGAGATTGGCATAAGTTCTT (SEQ ID NO.:3), or a sequence having at least 70% identity therewith (e.g., greater than 80%, 90%, 95%). In some embodiments, the reagents comprise an internal control target sequence. In some embodiments, the internal control target sequence is not homologous to an ASFV sequence. In some embodiments, the detecting comprises determining an amount of ASFV nucleic acid in the sample. In some embodiments, the detecting comprises detecting fluorescence associate with binding of a probe to the amplified ASFV nucleic acid after amplifying is completed. In some embodiments, the detecting comprises conducting a melt curve analysis between a probe and the amplified target nucleic acid. In some embodiments, the detecting differentiates ASVF from one or more or all of CSFV, PRRSV, PCV-2, PMWSV, SVDV, and VSV. In some embodiments, detecting differentiates ASVF from CSFV. In some embodiments, the detecting differentiates ASVF from PRRSV. In some embodiments, detecting differentiates ASVF from PCV-2. In some embodiments, detecting differentiates ASVF from PMWSV. In some embodiments, the detecting differentiates ASVF from SVDV. In some embodiments, detecting differentiates ASVF from VSV. In some embodiments, the reagents comprise Primesafe™II. In some embodiments, detecting identifies the strain of ASVF. In some embodiments, the reagents are contained within a reaction cartridge. In some embodiments, the reaction cartridge is configured to interact with a portable sample preparation and PCR instrument. In some embodiments, the portable sample preparation and PCR instrument comprises the Bio-Seeq Portable Veterinary Diagnostics Laboratory.

### BREIF DESCRIPTION OF THE DRAWINGS

FIG. 1 shows (a) detection of ASFV monoplex serial dilution using QSR670 probe; (b) melt derivative—QSR670 detection of ASFV monoplex serial dilution; (c) Detection of DNA control monoplex serial dilution using Cal Orange 560 probe; and (d) Melt derivative—Cal Orange 560 detection of DNA control monoplex serial dilution.

FIG. 2 shows ASFV duplex endpoint analysis at 35° C. using the Stratagene MxPro software. The full duplex was tested on a serial dilution of synthetic ASFV targets, and amplification was carried out for 50 cycles. All values are normalized to 70° C. with the baseline subtracted. (a) Endpoint detection of a serial dilution of ASFV synthetic target in the QSR670 channel. Fluorescence at endpoint is directly related to the starting concentration of target. Samples are detected down to approximately 1 copy/reaction. (b) Endpoint detection of 150 copies per reaction of the DNA control in the Cal Orange plotted as each of the ASFV target concentrations. All reactions contained the same copy number of DNA control target. Endpoint analysis shows all samples giving similar fluorescence.

FIG. 3 shows a) monoplex test of three clinical samples in QSR670 Channel. Ben97/1 was extracted from spleen tissue, Ken06 was extracted from tonsil tissue, and E75 was extracted from liver tissue. The ASFV monoplex was also tested against a negative control containing only porcine DNA, and two positive standard controls. NTCs contained no DNA. The threshold (dotted line) was set at 0.2 normalized fluorescent units. (b) ASFV clinical strain Ben97/1 dilution series in duplex endpoint format. The ASFV duplex was tested against a Ben97/1 clinical sample of ASFV that had been serially diluted to approximately 1 copy/μl (10-5). All values are normalized to 70° C. with the baseline subtracted.

FIG. 4 shows ASFV duplex detection of multiple ASFV strains at end-point. Fourteen viral DNA strains were tested and all showed a positive signal at 40° C. Differences in fluorescence reflect differences in DNA concentration. All data have been normalized to 70° C. with the background subtracted.

### Definitions

A “molecular beacon probe” is a single-stranded oligonucleotide, typically 25 to 35 bases-long, in which the bases on the 3′ and 5′ ends are complementary forming a “stem,” typically for 5 to 8 base pairs. In certain embodiments, the molecular beacons employed have stems that are exactly 2 or 3 base pairs in length. A molecular beacon probe forms a hairpin structure at temperatures at and below those used to anneal the primers to the template (typically below about 60° C.). The double-helical stem of the hairpin brings a fluorophore (or other label) attached to the 5′ end of the probe very close to a quencher attached to the 3′ end of the probe. The probe does not fluoresce (or otherwise provide a signal) in this conformation. If a probe is heated above the temperature needed to melt the double stranded stem apart, or the probe is allowed to hybridize to a target oligonucleotide that is complementary to the sequence within the single-strand loop of the probe, the fluorophore and the quencher are separated, and the fluorophore fluoresces in the resulting conformation. Therefore, in a series of PCR cycles the strength of the fluorescent signal increases in proportion to the amount of the beacon hybridized to the amplicon, when the signal is read at the annealing temperature. Molecular beacons with different loop sequences can be conjugated to different fluorophores in order to monitor increases in amplicons that differ by as little as one base (Tyagi, S. and Kramer, F. R. (1996), Nat. Biotech. 14:303 308; Tyagi, S. et al., (1998), Nat. Biotech. 16: 49 53; Kostrikis, L. G. et al., (1998), Science 279: 1228 1229; all of which are herein incorporated by reference).

As used herein, the term “amplicon” refers to a nucleic acid generated using primer pairs, such as those described herein. The amplicon is typically single-stranded DNA (e.g., the result of asymmetric amplification), however, it may be RNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. In certain embodiments, the type of amplification is asymmetric PCR (e.g., LATE-PCR) which is described in, for example, U.S. Pat. No. 7,198,897 and Pierce et al., PNAS, 2005, 102(24):8609-8614, both of which are herein incorporated by reference in their entireties.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/15=1.0 or 100% sequence identity with 75% of the 20 nucleobase primer. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, the term “hybridization” or “hybridize” is used in reference to the pairing of complementary nucleic acids. The strength of hybridization is expressed by the melting temperature, or effective melting temperature of hybridized nucleic acids. Melting temperature is influenced by such factors as the degree of complementary between the nucleic acids, stringency of the conditions involved, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide with a 3′OH, whether occurring naturally as in a purified restriction digest or produced synthetically, that is capable of forming a short double-stranded DNA/DNA or DNA/RNA hybrid on a longer template strand for initiation of synthesis via primer extension under permissive conditions (e.g., in the presence of nucleotides and an inducing agent such as a biocatalyst (e.g., a DNA polymerase or the like) and at a suitable temperature, pH, and ion composition). The primer is typically single stranded for maximum efficiency in amplification, but may alternatively be double stranded or partially double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

In some embodiments, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, etc.) from one or more bioagents, such as ASFV. Samples can include, for example, blood, saliva, urine, feces, anorectal swabs, vaginal swabs, cervical swabs, and the like. Sample may also be environmental samples, such as soil, water, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, electrical labels, molecular weight labels, phosphorescent or fluorogenic moieties; and fluorescent dyes alone or in combination with moieties that can suppress (“quench”) or shift emission spectra by fluorescence resonance energy transfer (FRET). FRET is a distance-dependent interaction between the electronic excited states of two molecules (e.g., two dye molecules, or a dye molecule and a non-fluorescing quencher molecule) in which excitation is transferred from a donor molecule to an acceptor molecule without emission of a photon. (Stryer et al., 1978, Ann. Rev. Biochem., 47:819; Selvin, 1995, Methods Enzymol., 246:300, each incorporated herein by reference). As used herein, the term “donor” refers to a fluorophore that absorbs at a first wavelength and emits at a second, longer wavelength. The term “acceptor” refers to a moiety such as a fluorophore, chromophore, or quencher that has an absorption spectrum that overlaps the donor's emission spectrum, and that is able to absorb some or most of the emitted energy from the donor when it is near the donor group (typically between 1-100 nm). If the acceptor is a fluorophore, it generally then re-emits at a third, still longer wavelength; if it is a chromophore or quencher, it then releases the energy absorbed from the donor without emitting a photon. In some embodiments, changes in detectable emission from a donor dye (e.g. when an acceptor moiety is near or distant) are detected. In some embodiments, changes in detectable emission from an acceptor dye are detected. In some embodiments, the emission spectrum of the acceptor dye is distinct from the emission spectrum of the donor dye such that emissions from the dyes can be differentiated (e.g., spectrally resolved) from each other.

Labels may provide signals detectable by fluorescence (e.g., simple fluorescence, FRET, time-resolved fluorescence, fluorescence polarization, etc.), radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral.

“T_{M},” or “melting temperature,” of an oligonucleotide describes the temperature (in degrees Celsius) at which 50% of the molecules in a population of a single-stranded oligonucleotide are hybridized to their complementary sequence and 50% of the molecules in the population are not-hybridized to the complementary sequence. The T_{M} of a primer or probe can be determined empirically by means of a melting curve. In some cases it can also be calculated. For the design of symmetric and asymmetric PCR primer pairs, balanced T_{M}'s are generally calculated by one of the three methods, that is, the “% GC”, or the “2(A+T) plus 4 (G+C)”, or “Nearest Neighbor” formula at some chosen set of conditions of monovalent salt concentration and primer concentration. In the case of Nearest Neighbor calculations the T_{M}'s of both primers will depend on the concentrations chosen for use in calculation or measurement, the difference between the T_{M}'s of the two primers will not change substantially as long as the primer concentrations are equimolar, as they normally are with respect to PCR primer measurements and calculations. T_{M}[1] describes the calculated T_{M} of a PCR primer at particular standard conditions of 1 micromolar (1 uM=10⁻⁶M) primer concentration, and 0.07 molar monovalent cations. In this application, unless otherwise stated, T_{M}[1] is calculated using Nearest Neighbor formula, T_{M}=ΔH/(ΔS+R ln(C/2))−273.15+12 log [M]. This formula is based on the published formula (Le Novere, N. (2001), “MELTING, Computing the Melting Temperature of Nucleic Acid Duplex,” Bioinformatics 17: 1226 7). ΔH is the enthalpy and ΔS is the entropy (both ΔH and ΔS calculations are based on Allawi and SantaLucia, 1997), C is the concentration of the oligonucleotide (10⁻⁶M), R is the universal gas constant, and [M] is the molar concentration of monovalent cations (0.07). According to this formula the nucleotide base composition of the oligonucleotide (contained in the terms ΔH and ΔS), the salt concentration, and the concentration of the oligonucleotide (contained in the term C) influence the T_{M}. In general for oligonucleotides of the same length, the T_{M} increases as the percentage of guanine and cytosine bases of the oligonucleotide increases, but the T_{M} decreases as the concentration of the oligonucleotide decreases. In the case of a primer with nucleotides other than A, T, C and G or with covalent modification, T_{M}[1] is measured empirically by hybridization melting analysis as known in the art.

“T_{M}[0]” means the T_{M} of a PCR primer or probe at the start of a PCR amplification taking into account its starting concentration, length, and composition. Unless otherwise stated, T_{M}[0] is the calculated T_{M} of a PCR primer at the actual starting concentration of that primer in the reaction mixture, under assumed standard conditions of 0.07 M monovalent cations and the presence of a vast excess concentration of a target oligonucleotide having a sequence complementary to that of the primer. In instances where a target sequence is not fully complementary to a primer it is important to consider not only the T_{M}[0] of the primer against its complements but also the concentration-adjusted melting point of the imperfect hybrid formed between the primer and the target. In this application, T_{M}[0] for a primer is calculated using the Nearest Neighbor formula and conditions stated in the previous paragraph, but using the actual starting micromolar concentration of the primer. In the case of a primer with nucleotides other than A, T, C and G or with covalent modification, T_{M}[0] is measured empirically by hybridization melting analysis as known in the art.

As used herein superscript X refers to the Excess Primer, superscript L refers to the Limiting Primer, superscript A refers to the amplicon, and superscript P refers to the probe.

T_{M}^{A} means the melting temperature of an amplicon, either a double-stranded amplicon or a single-stranded amplicon hybridized to its complement. In this application, unless otherwise stated, the melting point of an amplicon, or T_{M}^{A}, refers to the T_{M} calculated by the following % GC formula: Tₘ^{A}=81.5+0.41(% G+% C)−500/L+16.6 log [M]/(1+0.7 [M]), where L is the length in nucleotides and [M] is the molar concentration of monovalent cations.

T_{M}[0]^{P} refers to the concentration-adjusted melting temperature of the probe to its target, or the portion of probe that actually is complementary to the target sequence (e.g., the loop sequence of a molecular beacon probe). In the case of most linear probes, T_{M}[0]^{P} is calculated using the Nearest Neighbor formula given above, as for T_{M}[0], or preferably is measured empirically. In the case of molecular beacons, a rough estimate of T_{M}[0]^{P} can be calculated using commercially available computer programs that utilize the % GC method, see Marras, S. A. et al. (1999) “Multiplex Detection of Single-Nucleotide Variations Using Molecular Beacons,” Genet. Anal. 14:151 156, or using the Nearest Neighbor formula, or preferably is measured empirically. In the case of probes having non-conventional bases and for double-stranded probes, T_{M}[0]^{P} is determined empirically.

C_{T} means threshold cycle and signifies the cycle of a real-time PCR amplification assay in which signal from a reporter indicative of amplicons generation first becomes detectable above background. Because empirically measured background levels can be slightly variable, it is standard practice to measure the C_{T} at the point in the reaction when the signal reaches 10 standard deviations above the background level averaged over the 5-10 preceding thermal cycles.

### DETAILED DESCRIPTION

Provided herein are compositions and methods for the detection and analysis of African swine fever virus (ASFV). In particular, kits, compositions, and methods employing LATE-PCR reagents and processes for the detection and analysis of ASFV are provided. Although the the compositions and methods described herein are not limited to LATE-PCR. LATE-PCR is an advanced form of asymmetric PCR which is within the scope of the embodiments provided herein.

In some embodiments, provided herein are kits, compositions, and methods for ASFV detection based on Linear-After-The-Exponential (LATE) PCR (Pierce et al. 2007, herein incorporated by reference in its entirety), an advanced form of asymmetric PCR, that allows for rapid and sensitive detection at endpoint. In some embodiments, provided herein are kits, compositions, and methods for ASFV detection with Primesafe™II (Rice et al. 2007, herein incorporated by reference in its entirety), a PCR additive that maintains the fidelity of amplification over a broad range of target concentrations by suppressing mis-priming throughout the reaction. In some embodiments, kits, compositions, and methods are provided utilizing both LATE PCR and Primesafe™II. LATE-PCR assays reliably generate abundant single-stranded amplicons that can readily be detected in real-time and/or characterized at end-point using probes. In some embodiments, the assay functions as a duplex with an internal DNA control. Experiments conducted during the development of some embodiments described herein demonstrated that the detection limit of the duplex assay was determined to be approximately one genome copy per reaction with both synthetic target and clinical samples. Testing of this system gave a positive signal for fourteen different ASFV strains, as well as three clinical samples. It was also specific to ASFV, testing negative against similar viruses. Thus, some embodiments provide highly informative, sensitive, and robust results not provided by existing commercial technology used to detect and analyze ASFV.

The LATE-PCR assays described here can be used on both standard laboratory equipment or in the Bio-Seeq Portable Veterinary Diagnostics Laboratory, a portable sample preparation and PCR instrument built by Smiths Detection. This device is specifically engineered for use in the field with a minimum of operator training. It includes an automated sample preparation unit that carries out sample preparation and LATE-PCR analysis on site in a matter of hours. Individual sample preparation units for the Bio-SeeqII, as well as the entire machine can be immersed in disinfectants (Virkon or Fam30) so as to ensure that virus is not transported away from the site of field testing.

Linear-After-The-Exponential-PCR (LATE-PCR) is an advanced form of asymmetric PCR. By applying this principle, a powerful assay for ASFV detection and identification is provided. The results indicate that the LATE-PCR assay is capable of detecting below 10 viral genome copies in the clinical specimens. Since the assay is designed to be used in either laboratory settings or in a portable PCR machine (Bio-Seeq Portable Veterinary Diagnostics Laboratory; Smiths Detection, Watford UK), the LATE-PCR provides a robust and unparalleled tool for the diagnosis of AFSV both in diagnostic institutes and in the field.

When using LATE-PCR, each reaction produces large amounts of specific, single-stranded DNA, which can then be probed with a sequence-specific probe. When tested against synthetic targets, the assay proved to be specific and effective even at low target numbers. Indeed, this assay generated robust specific signals down to approximately 1 molecule/reaction. The internal DNA control present in the assay is also specific and sensitive at low copy number. Experiments conducted with the control showed that there are no detectable nonspecific interactions or false positives produced by the assay.

In certain embodiments, the assays described herein employ primer pairs to amplify target nucleic acid sequences. The methods described herein are not limited by the type of amplification that is employed. In certain embodiments, asymmetric PCR is employed, such as LATE-PCR.

PCR is a repeated series of steps of denaturation, or strand melting, to create single-stranded templates; primer annealing; and primer extension by a thermally stable DNA polymerase. During the course of the reaction the times and temperatures of individual steps in the reaction may remain unchanged from cycle to cycle, or they may be changed at one or more points in the course of the reaction to promote efficiency or enhance selectivity. In addition to the pair of primers and target nucleic acid a PCR reaction mixture typically contains each of the four deoxyribonucleotide 5′ triphosphates (dNTPs) at equimolar concentrations, a thermostable polymerase, a divalent cation, and a buffering agent. A reverse transcriptase is included for RNA targets, unless the polymerase possesses that activity. The volume of such reactions is typically 25-100 ul. Multiple target sequences can be amplified in the same reaction. In the case of cDNA amplification, PCR is preceded by a separate reaction for reverse transcription of RNA into cDNA, unless the polymerase used in the PCR possesses reverse transcriptase activity. The number of cycles for a particular PCR amplification depends on several factors including: a) the amount of the starting material, b) the efficiency of the reaction, and c) the method and sensitivity of detection or subsequent analysis of the product.

Ideally, each strand of each amplicon molecule binds a primer at one end and serves as a template for a subsequent round of synthesis. The rate of generation of primer extension products, or amplicons, is thus generally exponential, theoretically doubling during each cycle. The amplicons include both plus (+) and minus (−) strands, which hybridize to one another to form double strands. To differentiate typical PCR from special variations described herein, typical PCR is referred to as “symmetric” PCR. Symmetric PCR thus results in an exponential increase of one or more double-stranded amplicon molecules, and both strands of each amplicon accumulate in equal amounts during each round of replication. The efficiency of exponential amplification via symmetric PCR eventually declines, and the rate of amplicon accumulation slows down and stops. Kinetic analysis of symmetric PCR reveals that reactions are composed of: a) an undetected amplification phase (initial cycles) during which both strands of the target sequence increase exponentially, but the amount of the product thus far accumulated is below the detectable level for the particular method of detection in use; b) a detected amplification phase (additional cycles) during which both strands of the target sequence continue to increase in parallel and the amount of the product is detectable; c) a plateau phase (terminal cycles) during which synthesis of both strands of the amplicon gradually stops and the amount of product no longer increases. Symmetric reactions slow down and the rate plateaus when the total amount of the double stranded DNA in the reaction becomes sufficiently high to bind all of the polymerase—thereby making it unavailable to bind to the primers on the template strand. Typically reactions are run long enough to guarantee accumulation of a detectable amount of product, without regard to the exact number of cycles needed to accomplish that purpose.

In certain embodiments, an amplification method is used that is known as “Linear-After-The Exponential PCR” or, for short, “LATE-PCR.” LATE-PCR is a non-symmetric PCR method; that is, it utilizes unequal concentrations of primers and yields single-stranded primer-extension products, or amplicons. LATE-PCR includes innovations in primer design, in temperature cycling profiles, and in hybridization probe design. Being a type of PCR process, LATE-PCR utilizes the basic steps of strand melting, primer annealing, and primer extension by a DNA polymerase caused or enabled to occur repeatedly by a series of temperature cycles. In the early cycles of a LATE-PCR amplification, when both primers are present, LATE-PCR amplification amplifies both strands of a target sequence exponentially, as occurs in conventional symmetric PCR. LATE-PCR then switches to synthesis of only one strand of the target sequence for additional cycles of amplification. In certain real-time LATE-PCR assays, the limiting primer is exhausted within a few cycles after the reaction reaches its C_{T} value, and in the certain assays one cycle after the reaction reaches its C_{T} value. As defined above, the C_{T} value is the thermal cycle at which signal becomes detectable above the empirically determined background level of the reaction. Whereas a symmetric PCR amplification typically reaches a plateau phase and stops generating new amplicons by the 50th thermal cycle, LATE-PCR amplifications do not plateau, because the do not continue to accumulate double-stranded products, and thus continue to generate single-stranded amplicons well beyond the 50th cycle, even through the 100th cycle. LATE-PCR amplifications and assays typically include at least 60 cycles, preferably at least 70 cycles when small (10,000 or less) numbers of target molecules are present at the start of amplification.

With certain exceptions, the ingredients of a reaction mixture for LATE-PCR amplification are generally the same as the ingredients of a reaction mixture for a corresponding symmetric PCR amplification. The mixture typically includes each of the four deoxyribonucleotide 5′ triphosphates (dNTPs) at equimolar concentrations, a thermostable polymerase, a divalent cation, and a buffering agent. As with symmetric PCR amplifications, it may include additional ingredients, for example reverse transcriptase for RNA targets. Non-natural dNTPs may be utilized. For instance, dUTP can be substituted for dTTP and used at 3 times the concentration of the other dNTPs due to the less efficient incorporation by Taq DNA polymerase.

In certain embodiments, the starting molar concentration of one primer, the “Limiting Primer,” is less than the starting molar concentration of the other primer, the “Excess Primer.” The ratio of the starting concentrations of the Excess Primer and the Limiting Primer is generally at least 5:1, preferably at least 10:1, and more preferably at least 20:1. The ratio of Excess Primer to Limiting Primer can be, for example, 5:1 . . . 10:1, 15:1 . . . 20:1 . . . 25:1 . . . 30:1 . . . 35:1 . . . 40:1 . . . 45:1 . . . 50:1 . . . 55:1 . . . 60:1 . . . 65:1 . . . 70:1 . . . 75:1 . . . 80:1 . . . 85:1 . . . 90:1 . . . 95:1 . . . or 100:1 . . . 1000:1 . . . or more. Primer length and sequence are adjusted or modified, preferably at the 5′ end of the molecule, such that the concentration-adjusted melting temperature of the Limiting Primer at the start of the reaction, T_{M}[0]^{L}, is greater than or equal (plus or minus 0.5 degrees C.) to the concentration-adjusted melting point of the Excess Primer at the start of the reaction, T_{M}[0]^{X}. Preferably the difference (T_{M}[0]^{L}−T_{M}[0]^{X}) is at least +3, and more preferably the difference is at least +5 degrees C.

Amplifications and assays according to embodiments of methods described herein can be performed with initial reaction mixtures having ranges of concentrations of target molecules and primers. LATE-PCR assays are particularly suited for amplifications that utilize small reaction-mixture volumes and relatively few molecules containing the target sequence, sometimes referred to as “low copy number.” While LATE-PCR can be used to assay samples containing large amounts of target, for example up to 10⁶ copies of target molecules, other ranges that can be employed are much smaller amounts, from to 1-50,000 copies, 1-10,000 copies and 1-1,000 copies. In certain embodiments, the concentration of the Limiting Primer is from a few nanomolar (nM) up to 200 nM. The Limiting Primer concentration is preferably as far toward the low end of the range as detection sensitivity permits.

Also provided are compositions (e.g., kits, kit components, systems, instruments, reaction mixtures) comprising one or more or all of the components useful, necessary, or sufficient for carrying out any of the methods described herein. In some embodiments, kits are provided containing one or more or all of the reagents.

### EXPEIMENTAL

### Example 1

### Compositions and Methods

LATE PCR assay: In some embodiments, the ASFV assay provides amplification of the VP72 gene of African Swine Fever Virus based on an alignment of 32 sequences from GenBank using ClustalW alignment software (http://www.ebi.ac.uk/Tools/clustalw2/index.html) and takes advantage of the production of ssDNA and large detection temperature space provided by LATE-PCR. The duplex assay includes an internal DNA control, which is a synthetic target of no known function, designed to be innocuous. Primer and probe design for both ASFV and the DNA control followed the criteria of LATE-PCR outlined by Sanchez et al. (2004), and Pierce et. al (2005). Fluorescent reads are acquired using endpoint analysis after PCR amplification. Amplification of the correct product was verified via melt analysis. Verification can be conducted by any suitable method known to those of skill in the art.

Primers, probes, and targets: The ASFV Limiting primer (LP), Excess primer (XP) and the fluorescent probe were designed to amplify and detect a 247 bp region of pathogenic isolate E70 (GenBank Accession AY578692) using LATE-PCR design criteria. The LP was designed to have a melting temperature (Tₘ) higher than the XP, resulting in efficient exponential amplification of a double-stranded amplicon followed by abrupt switching to linear amplification of a single-strand when the LP runs out. The probe had a melting temperature of 55.5° C. to prevent interference with primer binding and extension during the annealing step, 58.0° C., of amplification, but does bind at end-point when the temperature is dropped (Sanchez et al. 2004). The design for the DNA control primers was originally based on the Xist gene expressed in female mouse embryos (Hartshorn et al., 2007). The primers were modified to match LATE-PCR primer criteria with melting temperatures close to the ASFV primer sequences. The DNA control probe is a synthetic sequence with no known origin designed to fit LATE-PCR probe criteria. All sequences are shown in Table 1. The ASFV probe was designed with a single G/T mismatch to the original target to reduce the effects of a hairpin in the probe structure. Nonspecific interactions were avoided based on Visual OMP (version 6.6.0) software (DNA Software, Inc., Ann Arbor, Mich.). This program was also used to calculate melting temperatures at the initial concentrations of the primers and probes.

TABLE 1
Sequences and Melting Temperatures of ASFV and DNA Control Primers^{a} and Probes

| Name | Sequence (5′→3′) | Modification | Tₘ^{b} |
|---|---|---|---|
| ASFV LP | CTGATACGTGTCCATAAAACGCAGGTGAC | None | 69.3° C. |
| | (SEQ ID NO.: 1) | | |
| ASFV XP | CTGGAAGAGCTGTATCTCTATCCTG | None | 67.1° C. |
| | (SEQ ID NO.: 2) | | |
| ASFV Probe | AACGAGATTGGCATAAGTTCTT (SEQ ID NO.: 3) | 5′ Quasar 670; 3′ | 55.5° C. |
| | | BHQ2 | |
| DNA Control | CGTTAACTTGTCGAGCCTACGTGTTCTACTCC | None | 71.1° C. |
| LP | (SEQ ID NO.: 4) | | |
| DNA Control | GAGCTGAACACCTACTACTTGATCT | None | 67.4° C. |
| XP | (SEQ ID NO.: 5) | | |
| DNA Control | AACGACTCTTAATCACAGCTT (SEQ ID NO.: 6) | 5′ Cal Orange | 57.2° C. |
| Probe | | 560; 3′ BHQ1 | |

^{a}The DNA control primer pair was designed to be within one degree of the respective ASFV primers (ΔTₘ^{XP} < 1° C., ΔTₘ^{LP} < 1° C.). The ASFV probe was modified with a 5′ Quasar 670 fluor (QSR670) and a 3′ Black Hole Quencher 2 (BHQ2). The DNA control probe was modified with a Cal Orange 560 fluor and a BHQ1. All melting temperatures were calculated by Visual OMP.
^{b}Tₘ = melting temperature at the starting concentration

Experiments were conducted during development of embodiments described herein to test the assay against a truncated, synthetic ssDNA target. The duplex reaction included the synthetic ssDNA control target. All synthetic targets and primers were ordered from Sigma-Aldrich (St. Louis, Mo., USA). The sequences for the synthesized oligonucleotide targets are shown in Table 2.

TABLE 2
Sequences and Melting Temperatures of ASFV and DNA Control
Synthetic Test Targets^{a}

| Name | Sequence | Tₘ^{b} |
|---|---|---|
| ASFV | CTGGAAGAGCTGTATCTCTATCCTGAAAGCTTACATGTCCGAACTT | 83.2° C. |
| Target | GTGCCAATCTCGGTGTTGAGGTGTGGGTCACCTGCGTTTTATGGAC | |
| | ACGTATCAG (SEQ ID NO.: 7) | |
| DNA | GAGCTGAACACCTACTACTTGATCTTACTTGCTGTGATTAAGAGTC | 79° C. |
| Control | GAACATGGGAGTAGAACACGTAGGCTCGACAAGTTAACG | |
| Target | (SEQ ID NO.: 8) | |

^{a}Synthetic test targets were manufactured as single-stranded molecules.
bThe double-stranded amplicon melting temperatures were calculated by Visual OMP. The real viral test target for ASFV is 247 bp.

Assay Composition: Each reaction was run in a final volume of 25 μl and contained the following reagents: 1× PCR buffer (Invitrogen, Cat. No: 60684-050), 3 mM MgCl₂, 250 μM dNTPs, 50 nM ASFV Limiting Primer, 1 μM ASFV Excess Primer, 50 nM DNA Control Limiting Primer, 1 μM DNA Control Excess Primer, 100 nM ASFV Probe with a 5′ QSR670 fluor and a 3′ Black Hole Quencher 2, 100 nM DNA Control probe with a 5′ Cal Orange 560 fluor and a 3′ Black Hole Quencher 1 (Biosearch Technologies, Novato, Calif., USA), 300 nM Primesafe™II (Rice et al. 2007) and 2.5 units of antibody-complexed Platinum® TFI exo (−) DNA polymerase (Invitrogen, Carlsbad, Calif.).

ASFV Samples and Handling: DNA from ASFV DNA reference samples (Table 3) was extracted directly from primary cell cultures (leukocytes and/or alveolar macrophages) using a nucleic acid extraction kit (Nucleospin/Machery-Nagel-Cultek) following the manufacturer's procedures. The DNA was then concentrated by ethanol precipitation: 1/10 volume of 3M NaOAc and 3 volumes ethanol were added to the DNA solution then left overnight at −70° C. The solution was spun in a microcentrifuge for 10 minutes to pellet the DNA, then washed with 70% ethanol and spun for another 10 minutes. The DNA was air-dried and resuspended in a final volume of 100 μl of distillate RNAse-free water. Each sample was diluted 1:10 in water before testing.

TABLE 3
ASFV strains used in the study.

| | | Origin/ | Isolation | Institute |
|---|---|---|---|---|
| Isolate | Name | Source | Country | Provider |
| Mozambique | Moz64 | Pig | Mozambique | CISA-INIA |
| 1964 | | | | |
| Angola 1972 | Ang72 | Pig | Angola | CISA-INIA |
| Chalaswa 1983 | MwLil | Tick, pig | Malawi | CISA-INIA |
| | 20/1 | pen | | |
| Cape Verde 1997 | CV97 | Pig | Cape Verde | CISA-INIA |
| Hoima 2003 | Ug03H | Pig | Uganda | CISA-INIA |
| Kenya 2006 | Ken06.B1 | Pig | Kenya | CISA-INIA |
| Kenya 2007 | Ken07.Eld1 | Pig | Kenya | CISA-INIA |
| Burkina Faso | BF07 | Pig | Burkina Faso | CISA-INIA |
| 2007 | | | | |
| Pontevedra 1970 | E70 | Pig | Spain | CISA-INIA |
| Badajoz 1971 | Ba71V | Vero cell | Spain | CISA-INIA |
| | | adapted | | |
| | | pig isolate | | |
| Lerida 1975 | E75 | Pig | Spain | CISA-INIA |
| Lisbon 60 | L60 | Pig | Portugal | CISA-INIA |
| Sardinia 1988 | Ss88 | Pig | Italy | CISA-INIA |
| Port-au-Prince 81 | Haiti | Pig | Haiti | CISA-INIA |

Abbreviations:
CISA-INIA—Centro de Investigación en Sanidad Animal, del Instituto Nacional de Investigación y Tecnología Agraria y Alimentaria. Samples were generously provided by Dr. Carmina Gallardo.

Clinical Samples: Spleen, tonsil, and liver tissue samples from experimentally infected pigs were obtained (Table 4). DNA from the tissue samples was isolated by a Magnatrix 8000 extraction robot and MagAttract Virus Mini Kit protocol (Qiagen), according to the manufacturer's instructions. The nucleic acid from each sample was eluted in 100 μl of elution buffer and stored at −20° C. until use. The samples, together with two positive standards, and porcine DNA as positive and negative control, respectively, were tested in monoplex format, in the Rotor-Gene 3000 (Qiagen/Corbett Life Science, Valencia, Calif.) with the following thermal profile: 1 cycle at 95° C. for 3 minutes; 50 cycles of 95° C. for 10 sec, 58° C. for 15 sec, and 72° C. for 30 sec; and 1 cycle at 70° C. for 3 minutes, 50° C. for 3 minutes, 40° C. for 3 minutes, with fluorescence acquisition during the last cycle at 70° C., 50° C., and 40° C. in the Cy5 Channel (Source 625 nm, Detector 660 high pass filter nm).

TABLE 4
ASFV clinical samples used in the study

| | | | Virus titer of |
|---|---|---|---|
| Isolate | Tissue | Dpi | inoculum |
| Ben97/1 | Spleen | 7 | 10⁴ HAU/ml |
| Ken06 | Tonsil | 7 | 10⁵ HAU/ml |
| E75L2 | Liver | 10 | 10 HAU/ml |

Abbreviations:
HAU—Hemagglutinating unit
Dpi—days post infection

Conditions: Experiments were conducted during development of embodiments described herein in which PCR of synthetic targets was initially carried out in a Stratagene Mx3005P Sequence Detector (Stratagene, La Jolla, Calif.) with the following thermal profile: 1 cycle at 95° C. for 3 minutes; 50 cycles of 95° C. for 10 sec, 58° C. for 15 sec, and 72° C. for 30 sec; and 1 cycle at 70° C. for 3 minutes, 50° C. for 3 minutes, and 35° C. for 3 minutes with fluorescence acquisition during the last cycle at 70° C., 50° C., and 35° C. in the Quasar 670 and Cal Orange 560 channels. Experiments were run using endpoint analysis rather than real time to reduce nonspecific product.

PCR of viral DNA (cell culture and clinical samples) was carried out in a Rotor-Gene 3000 (Qiagen/Corbett Life Science, Valencia, Calif.) with the following thermal profile: 1 cycle at 95° C. for 3 minutes; 50 cycles of 95° C. for 10 sec, 58° C. for 15 sec, and 72° C. for 30 sec; and 1 cycle at 70° C. for 3 minutes, 50° C. for 3 minutes, 40° C. for 3 minutes, with fluorescence acquisition during the last cycle at 70° C., 50° C., and 40° C. in the Cy5 Channel (Source 625 nm, Detector 660 high pass filter nm) and JOE channel (Source 530 nm, Detector 555 nm). The lowest detection temperature is 40° C. due to the temperature limitations of the Rotor-Gene thermocycler.

Sensitivity determination and PCR efficiency: A series of dilutions of known concentration of the synthetic ASFV target monoplex were tested. Dilutions ranged from 10⁹ target copies/reaction to approximately 1 copy/reaction Target samples were prepared in TE buffer (10 mMTris-HCl, pH 8.0, 1 mMEDTA) containing 10 μg/ml salmon sperm DNA (Ambion, Austin, Tex., USA) to assure a constant amount of nucleic acids in the diluted samples. The number of copies in the stock solution was determined using the molarity of the template and Avogadro's formula. A standard curve was generated, and PCR efficiency was calculated using integrated Rotor-Gene 3000 instrument software. Dilutions were tested in real time format with the following thermal profile: 1 cycle at 95° C. for 3 minutes; 50 cycles of 95° C. for 10 sec, 58° C. for 15 sec, 72° C. for 30 sec, and 45° C. for 20 sec reading at 45° C. Dilutions were also tested at end point.

Range of detection and specificity tests: Experiments were conducted during development of embodiments described herein to determine the range of detection of the assay. 1:10 dilutions of the ASFV DNA samples were tested. To determine the specificity of the assay, it was also tested against seven viruses with similar symptoms to ASFV (Table 5).

TABLE 5
Other viruses used to test specificity of ASFV assay

| Virus | Strain/Serotype | Tissue | Source | Signal |
|---|---|---|---|---|
| CSFV | Alfort | Cell culture | SVA | Negative |
| PRRSV | European strain, | Serum | UCM | Negative |
| | SP2777 | | | |
| PCV-2 | Stoon-1010 | Plasmid | SVA | Negative |
| PMWSV | na | Peribronchial | CVI | Negative |
| | | Lymph nodes | | |
| SVDV | HKN 1/80 | Cell culture | IAH | Negative |
| VSV | Indiana-1 (Indiana C, | Cell culture | IAH | Negative |
| | 1942) | | | |
| VSV | New Jersey, Colorado | Cell culture | IAH | Negative |
| | 1984 | | | |

Abbreviations:
CSFV—Classical swine fever virus
PRRSV—Porcine respiratory and reproductive syndrome virus
PCV-2—Porcine circovirus 2
PMWSV—Porcine multisystemic wasting syndrome virus
SVDV—Swine vesicular disease virus
VSV—Vesicular stomatitis virus
SVA—National Veterinary Institute, Uppsala, Sweden
UCM—Universidad Complutense de Madrid, Madrid, Spain
CVI—Central Veterinary Institute, Budapest, Hungary
IAH—Institute for Animal Health, Pirbright, UK
na—not available

### Example 2

### Experimental Results

Experiments were conducted during development of embodiments described herein to design and verify a novel assay for detection of African swine fever virus (ASFV) based on Linear-After-The-Exponential Polymerase Chain Reaction (LATE-PCR). Because of the properties of LATE-PCR, each reaction produces large amounts of specific, single-stranded DNA, which can then be probed with a sequence-specific probe. When tested against synthetic targets, the assay proved to be specific and effective even at low target numbers. Indeed, this assay generated robust specific signals down to approximately 1 molecule/reaction. The internal DNA control present in the new assay is also specific and sensitive at low copy number. Results show the DNA control signal appearing only in the Cal Orange 560 channel. This indicates there are no nonspecific interactions or false positives produced by the assay.

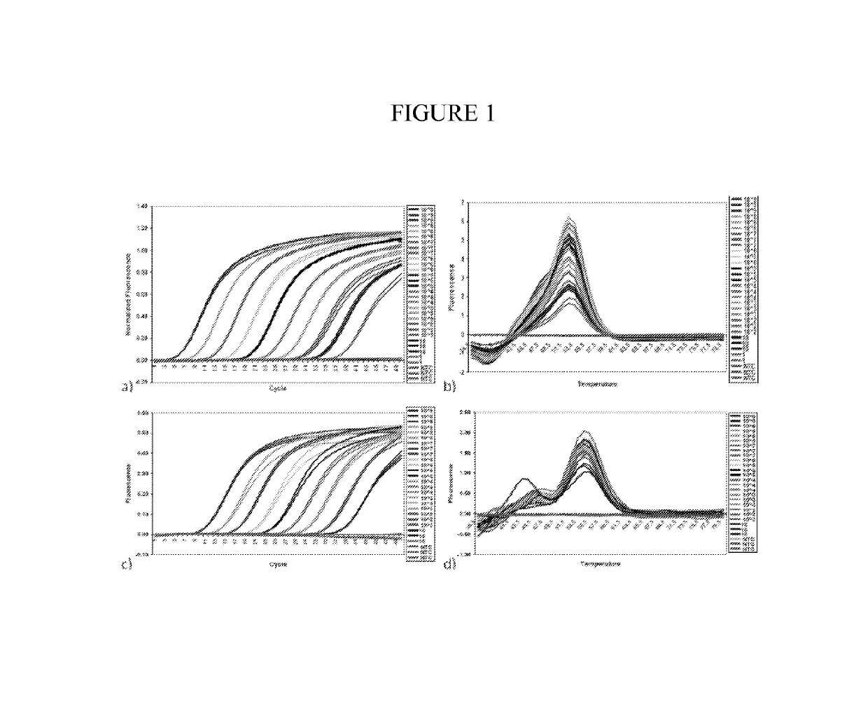

Assay optimization: The LATE-PCR assay was initially constructed and optimized in two separate monoplex reactions using synthetic ASFV and DNA control targets (SEE FIGS. 1A and 1C, respectively). The optimization and testing of synthetic targets was carried out in both a Rotor-Gene 3000 thermocycler and a Stratagene Mx3005P Sequence Detector.

Experiments were conducted during development of embodiments described herein to determine the efficiency and sensitivity of each monoplex. Serial dilutions from 10⁹ to 1 initial copies/reaction were carried out and detected in real time using a probe that hybridized to the accumulated product during a 45° C. step inserted into each thermal cycle after extension. A probe-target melt curve was constructed at end-point for the single-strand amplicons generated, and the 1^{st} derivative was taken to determine the Tₘ of all ASFV and all DNA control reactions (SEE FIGS. 1B and 1D, respectively). Products were detected in both sets of reactions at every dilution and replicate reactions were highly reproducible (SEE FIGS. 1A and 1C. All ASFV monoplex reactions generated very similar products with a dominant melt peak at 52.5° C. (SEE FIG. 1B). The melt derivative of the DNA control peaks at approximately 55.5° C. (SEE FIG. 1D. Reaction efficiencies of synthetic ASFV and DNA control targets based on a standard curve and calculated by Rotor-Gene 3000 instrument software were 93% and 96%, respectively (not shown).

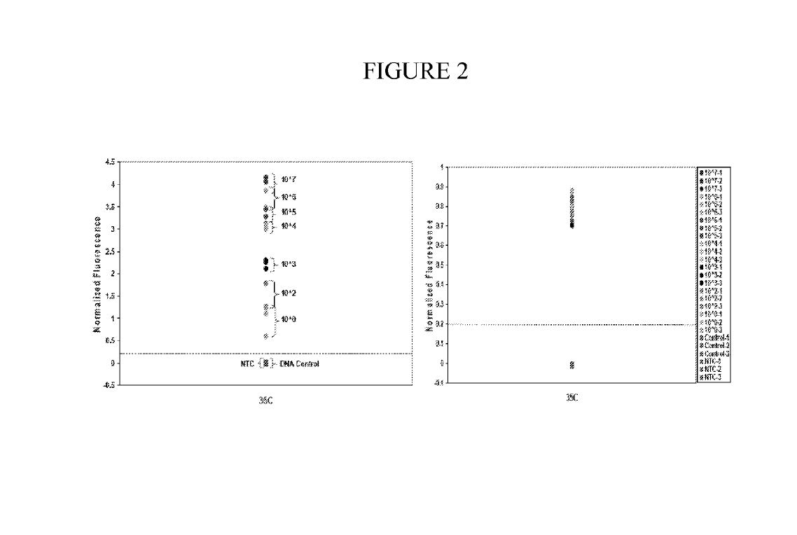

After optimization of the ASFV and DNA control monoplex reactions, the complete duplex reaction was tested at endpoint (SEE FIG. 2). The reaction comprised two ASFV primers, two DNA control primers, one ASFV probe, one DNA control probe and 300 nM PrimeSafe™II, to prevent nonspecific interactions during amplification. A serial dilution of the ASFV target was tested at endpoint after 50 cycles of amplification, reading in the QSR670 channel (SEE FIG. 2A). Each reaction reported contained 150 target copies/reaction of the DNA control. The ASFV amplification data are presented as a ratio of the fluorescence at 35° C. to the fluorescence at 70° C. with the baseline subtracted. A threshold value of 0.2 normalized fluorescent units above the negative control (normalized to equal 0) was chosen to establish a positive signal. Fluorescent ratios of the ASFV samples ranged from 0.6 (1 target/reaction) to 4.2 (10⁷ targets/reaction) normalized fluorescent units. The samples containing only the DNA control did not generate a signal in the QSR670 channel, but did do so in the Cal Orange 560 channel (SEE FIG. 2B). The resulting endpoint data are reported as a normalized value at 35° C. The threshold value for the DNA control was chosen as 0.2 for a positive signal. All of the DNA Control samples were detected in the Cal Orange 560 channel with signals ranging from 0.7 to 0.88 normalized fluorescent units.

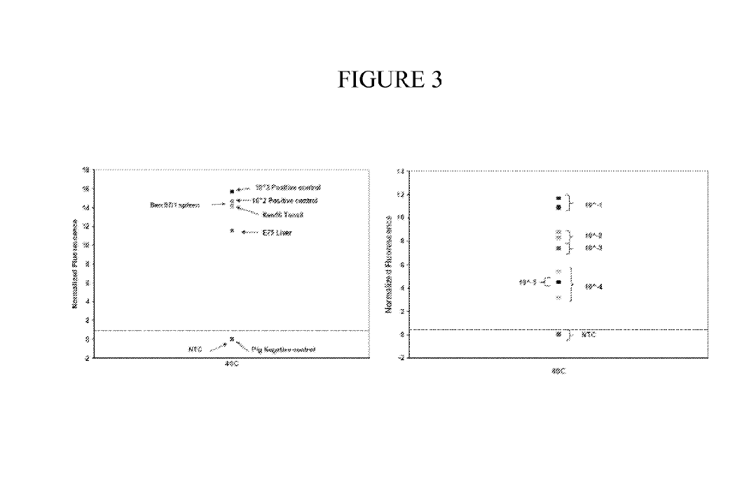

Sensitivity and specificity using viral DNA samples: Experiments were conducted during development of embodiments described herein to determine the sensitivity and specificity of the ASFV LATE-PCR assay. Both the monoplex and duplex were tested on clinical samples. Three samples, Ben97/1 from spleen tissue, Ken06 from tonsil tissue, and E75 from liver tissue, were tested in monoplex format, together with two positive standards, and porcine DNA as positive and negative controls, respectively (SEE FIG. 3A). All of the resulting data were normalized with the background subtracted. A threshold of 0.2 normalized fluorescent units above the negative control (normalized to 0) was chosen to establish a positive signal. All three clinical samples gave clear, positive signals. One sample, Ben97/1 was further tested in a serial dilution in duplex format (SEE FIG. 3B)). All dilutions were detected and one gene copy was achieved by a 10⁵ fold dilution.

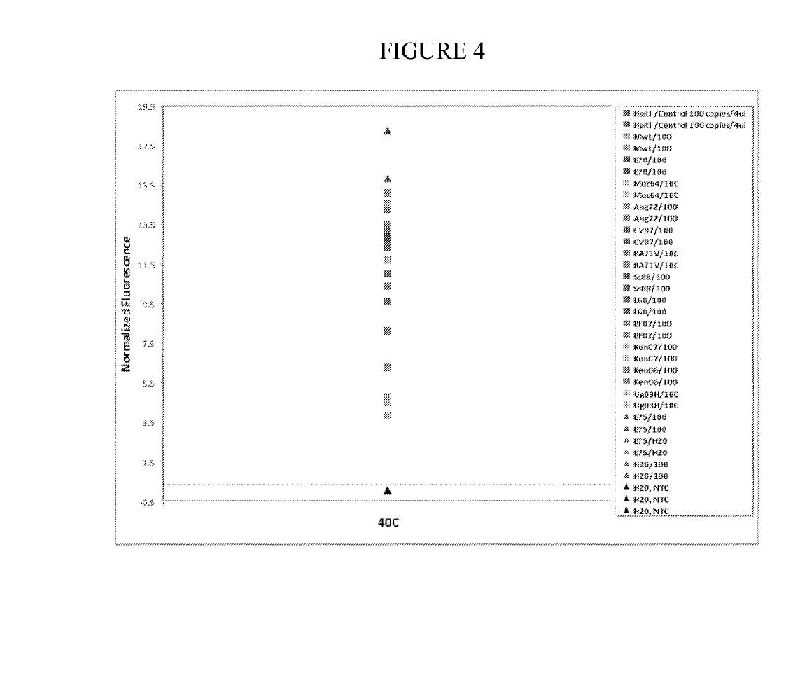

To determine the scope of the assay, the duplex was tested against a total of 14 different viral strains (Table 3) at the National Veterinary Institute in Uppsala, Sweden. The data from the 14 different strains were collected at end-point and were normalized to 70° C. with the background subtracted (SEE FIG. 4). The end-point data show a strong positive signal above the threshold (0.2 normalized fluorescent units) for all of the samples tested. Fluorescent values ranged from 3.8 to 15.1 normalized fluorescent units above the negative control (normalized to 0) with the positive control near 15 normalized fluorescent units.

The specificity of the LATE-PCR ASFV assay was determined by testing against seven ASFV-related viruses that cause similar symptoms to ASF and require the use of laboratory tests to differentiate them (Table 5). None of these viruses generated a positive signal, indicating that the assay is highly specific for ASFV.

Various modifications to, and variations of, the described methods and compositions of will be apparent to those skilled in the art without departing from the scope and spirit of compositions and methods provided. Although specific embodiments have been described and highlighted herein, it should be understood that the claims should not be unduly limited to such specific embodiments. Indeed, various modifications of the described modes for carrying the compositions and methods described herein that are obvious to those skilled in the relevant fields are intended to be within the scope of the following claims.

### REFERENCES

All publications and patents mentioned in the present application and/or listed below are herein incorporated by reference in their entireties.
Agüero, M., Fernandez, J., Romero, L. J., Zamora, M. J., Sánchez, C., Belák, S., Arias., Sánchez-Vizcaíno, J. M. (2004). “A highly sensitive and specific gel-based multiplex RT-PCR assay for the simultaneous and differential diagnosis of African swine fever and Classical swine fever in clinical samples.” Vet Res 35(5): 551-63.
Alcaraz, C., De Diego, M., Pastor, M. J., Escribano, J. M. (1990). “Comparison of a radioimmunoprecipitation assay to immunoblotting and ELISA for detection of antibody to African swine fever virus.” J Vet Diagn Invest 2(3): 191-6.
Anderson, E. C., Hutchings, G. H., Mukarati, N., Wilkinson, P. J. (1998). “African swine fever virus infection of the bushpig (Potamochoerus porcus) and its significance in the epidemiology of the disease.” Vet Microbiol 62(1): 1-15.
Bastos, A. D. S, Penrith, M. L., Crucière, C., Edrich, J. L., Hutchings, G., Roger, F., Coaucy-Hymann, Thomson, G. R. (2003). “Genotyping field strains of African swine fever virus by partial p72 gene characterisation.” Arch Virol 148(4): 693-706.
Bastos, A. D. S, Penrith, M. L., Macome, F., Pinto, F., Thomson, G. R (2004). “Co-circulation of two genetically distinct viruses in an outbreak of African swine fever in Mozambique: no evidence for individual co-infection.” Vet Microbiol 103(3-4): 169-82.
Blasco, R., Agüero, M., Almendral, J. M., Viñuela, E. (1989a). “Variable and constant regions in African swine fever virus DNA.” Virology 168(2): 330-8.
Blasco, R., de la Vega, I., Almazán, F., Agüero, M., Viñuela, E. (1989b). “Genetic variation of African swine fever virus: variable regions near the ends of the viral DNA.” Virology 173(1): 251-7.
Boinas, F. S., Hutchings, G. H., Dixon, L. K., Wilkinson, P. J. (2004). “Characterization of pathogenic and non-pathogenic African swine fever virus isolates from Ornithodoros erraticus inhabiting pig premises in Portugal.” J Gen Virol 85(Pt 8): 2177-87.
Dixon, L. K., Abrams, C. C., Bowick, G., Goatley, L. C., Kay-Jackson, P. C., Chapman, D., Liverani, E., Nix, R., Silk, R., Zhang, F. (2004). “African swine fever virus proteins involved in evading host defence systems.” Vet Immunol Immunopathol 100(3-4): 117-34.
Giammarioli, M., Pellegrini, C., Casciari, C., De Mia, G. M. (2008). “Development of a novel hot-start multiplex PCR for simultaneous detection of classical swine fever virus, African swine fever virus, porcine circovirus type 2, porcine reproductive and respiratory syndrome virus and porcine parvovirus.” Vet Res Commun 32(3): 255-62.
Hartshorn, C., Eckert, J. J., Hartung, O., Wangh, L. J. (2007). “Single-cell duplex RT-LATE-PCR reveals Oct4 and Xist RNA gradients in 8-cell embryos.” BMC Biotechnol 7: 87.
Hutchings, G. H. and Ferris, N. P. (2006). “Indirect sandwich ELISA for antigen detection of African swine fever virus: comparison of polyclonal and monoclonal antibodies.” J Virol Methods 131(2): 213-7.
ICTVdB (2006). 00.002.0.01.001. African swine fever virus. ICTVdB—The Universal Virus Database, version 4, Büchen-Osmond, C. (Ed), Columbia University, New York, USA.
King, D. P., Reid, S. M., Hutchings, G. H., Grierson, S. S., Wilkinson, P. J., Dixon, L. K., Bastos, A. D. S., Drew, T. W. (2003). “Development of a TaqMan® PCR assay with internal amplification control for the detection of African swine fever virus.” J Virol Methods 107(1): 53-61.
Kleiboeker, S. B. and Scoles, G. A. (2001). “Pathogenesis of African swine fever virus in Ornithodoros ticks.” Anim Health Res Rev 2(2): 121-8.
Malmquist, W. A. and Hay, D. (1960). “Hemadsorption and cytopathic effect produced by African Swine Fever virus in swine bone marrow and buffy coat cultures.” Am J Vet Res 21: 104-8.
McKillen, J., Hjertner, B., Millar, A., McNeilly, F., Belák, S., Adair, B., Allan, G. (2007). “Molecular beacon real-time PCR detection of swine viruses.” J Virol Methods 140(1-2): 155-65.
Montgomery, R. E. (1921). “On a form of swine fever occurring in British East Africa (Kenya Colony).” Journal of Comparative Pathology 34: 159-191.
Nix, R. J., Gallardo, C., Hutchings, G., Blanco, E., Dixon, L. K. (2006). “Molecular epidemiology of African swine fever virus studied by analysis of four variable genome regions.” Arch Virol 151(12): 2475-94.
Oura, C. A., Powell, P. P., Anderson, E., Parkhouse, R. M. E. (1998a). “The pathogenesis of African swine fever in the resistant bushpig.” J Gen Virol 79 (Pt 6): 1439-43.
Pierce, K. E., Sanchez, J. A., Rice, J. E., Wangh, L. J. (2005). “Linear-After-The-Exponential (LATE)-PCR: primer design criteria for high yields of specific single-stranded DNA and improved real-time detection.” Proc Natl Acad Sci USA 102(24): 8609-14.
Pierce, K. E. and Wangh, L. J. (2007). “Linear-after-the-exponential polymerase chain reaction and allied technologies. Real-time detection strategies for rapid, reliable diagnosis from single cells.” Methods Mol Med 132: 65-85.
Rice, J. E., Sanchez, J. A., Pierce, K. E., Reis, A. H. Jr., Osborne, A., Wangh, L. J. (2007). “Monoplex/multiplex linear-after-the-exponential-PCR assays combined with PrimeSafe™ and Dilute-‘N’-Go sequencing.” Nat. Protoc. 2(10): 2429-38.
Rodriguez-Sanchez B, Sanchez-Vizcaino J M, Uttenthal A, Rasmussen T B, Hakhverdyan M, King D P, Ferris N P, Ebert K, Reid S M, Kiss I, Brocchi E, Cordioli P, Hjerner B, McMenamy M, McKillen J, Ahmed J S, Belak S. (2008). Improved diagnosis for nine viral diseases considered as notifiable by the world organization for animal health. Transbound Emerg Dis. August; 55(5-6):215-25.
Rowlands, R. J., Michaud, V., Heath, L., Hutchings, G., Oura, C., Vosloo, W., Dwarka, R., Onashvili, T., Albina, E., Dixon, L. K. (2008). African Swine Fever Virus Isolate, Georgia, 2007. Emerg. Inf. Dis 14(12):1870-1874.
Sanchez, J. A., Pierce, K. E., Rice, J. E., Wangh, L. J. (2004). “Linear-after-the-exponential (LATE)-PCR: an advanced method of asymmetric PCR and its uses in quantitative real-time analysis.” Proc Natl Acad Sci USA 101(7): 1933-8.
Steiger, Y., Ackermann, M., Mettraux, C., Kihm, U. (1992). “Rapid and biologically safe diagnosis of African swine fever virus infection by using polymerase chain reaction.” J Clin Microbiol 30(1): 1-8.
Sumption, K. J., Hutchings, G. H., Wilkinson, P. J., Dixon, L. K. (1990). “Variable regions on the genome of Malawi isolates of African swine fever virus.” J Gen Virol 71 (Pt 10): 2331-40.
Zsak, L., Borca, M. V., Risatti, G. R., Zsak, A., French, R. A., Lu, Z., Kutish, G. F., Neilan, J. G., Callahan, J. D., Nelson, W. M., Rock, D. L. (2005). “Preclinical diagnosis of African swine fever in contact-exposed swine by a real-time PCR assay.” J Clin Microbiol 43(1): 112-9.

1.-72. (canceled)

73. A method for detecting or analyzing African swine fever virus (ASFV) in a sample, comprising: contacting one or more copies of viral genomic DNA isolated, or purified, or separated, or prepared from said sample with reagents for performing LATE-PCR, said reagents comprising a limiting primer and an excess primer pair for at least one target sequence, wherein the initial concentration-dependent melting temperature of said limiting primer is equal to or greater than the initial concentration-dependent melting temperature of the paired excess primer for corresponding ASFV target sequence; and amplifying said target sequence to generate specific double-stranded and single-stranded DNA products.

74. The method of claim 73, wherein the sample comprises an environmental or biological sample.

75. The method of claim 74, wherein the biological sample is a tissue or fluid taken from one or more wild or domesticated pigs.

76. The method of claim 73, wherein the virus is a strain of ASFV selected from the group consisting of Moz64, Ang72, MwLil 20/1, CV97, Ug03H, Ken06.B1, Ken07.Eld1, BF07, E70, Ba71V, E75, L60, Ss88, and Haiti.

77. The method of claim 73, wherein said target sequence includes a sequence of the ASFV VP72 gene.

78. The method of claim 73, wherein the reagents further comprise at least one fluorescent probe that hybridizes to said target sequence at a temperature at least 5° C. below the initial concentration dependent melting temperature of the limiting primer used to amplify said target sequence

79. The method of claim 78, wherein the fluorescent probe is a molecular beacon.

80. The method of claim 78, wherein the probe comprises SEQ ID NO.:3, or a sequence having at least 70% identity therewith.

81. The method of claim 73, wherein the reagents comprise an internal control target sequence that is not homologous to an ASFV sequence.

82. The method of claim 73, wherein detecting comprises determining the number of copies of viral genomic DNA in the sample.

83. The method of claim 73, wherein detecting differentiates ASVF from one or more of CSFV, PRRSV, PCV-2, PMWSV, SVDV, and VSV.

84. The method of claim 73, wherein detecting identifies the strain of ASVF.

85. A kit for detecting or analyzing African swine fever virus (ASFV) in a sample, comprising a limiting primer and an excess primer for LATE-PCR amplification of a target sequence of ASVF nucleic acid, wherein the initial concentration-dependent melting temperature of said limiting primer is equal to or greater than the initial concentration-dependent melting temperature of the corresponding excess primer for the ASFV target sequence.

86. The kit of claim 85, wherein the virus is a strain of ASFV selected from the group consisting of Moz64, Ang72, MwLil 20/1, CV97, Ug03H, Ken06.B1, Ken07.Eld1, BF07, E70, Ba71V, E75, L60, Ss88, and Haiti.

87. The kit of claim 85, wherein the said target sequence includes a sequence of the ASFV VP72 gene.

88. The kit of claim 85, further comprising at least one fluorescent probe that hybridizes to said target sequence at a temperature at least 5° C. below the initial concentration dependent melting temperature of the limiting primer used to amplify said target sequence

89. The kit of claim 88, wherein the fluorescent probe is a molecular beacon.

90. The kit of claim 88, wherein the probe comprises SEQ ID NO.:3, or a sequence having at least 70% identity therewith.

91. The kit of claim 85, further comprising an internal control target sequence that is not homologous to an ASFV sequence.

92. The kit of claim 85, wherein the reagents are contained within a reaction cartridge is configured to interact with a portable sample preparation and PCR instrument.


SEQUENCE LISTING (8 sequences)

SEQ ID NO: 1 - DNA, 29 bp, Artificial Sequence
  Synthetic
ctgatacgtgtccataaaacgcaggtgac

SEQ ID NO: 2 - DNA, 25 bp, Artificial Sequence
  Synthetic
ctggaagagctgtatctctatcctg

SEQ ID NO: 3 - DNA, 22 bp, Artificial Sequence
  Synthetic
aacgagattggcataagttctt

SEQ ID NO: 4 - DNA, 32 bp, Artificial Sequence
  Synthetic
cgttaacttgtcgagcctacgtgttctactcc

SEQ ID NO: 5 - DNA, 25 bp, Artificial Sequence
  Synthetic
gagctgaacacctactacttgatct

SEQ ID NO: 6 - DNA, 21 bp, Artificial Sequence
  Synthetic
aacgactcttaatcacagctt

SEQ ID NO: 7 - DNA, 101 bp, Artificial Sequence
  Synthetic
ctggaagagctgtatctctatcctgaaagcttacatgtccgaacttgtgccaatctcggtgttgaggtgtgggtcacctgcgttttatggacacgtatcag

SEQ ID NO: 8 - DNA, 85 bp, Artificial Sequence
  Synthetic
gagctgaacacctactacttgatcttacttgctgtgattaagagtcgaacatgggagtagaacacgtaggctcgacaagttaacg